(19) United States
(12) Patent Application Publication
(10) Pub. No.: US 2026/0245979 A1
(43) Pub. Date: Aug. 20, 2026

(54) ONIUM CATION WITH ETHER LINKAGES FOR ZINC BATTERIES

(71) Applicant: U.S. Army DEVCOM, Army Research Laboratory, Adelphi, MD (US)

(72) Inventors: Glenn R. Pastel, Silver Spring, MD (US); Kang Xu, Potomac, MD (US); Marshall A. Schroeder, Pasadena, MD (US); Travis P. Pollard, Rockville, MD (US); Oleg A. Borodin, Laurel, MD (US); Zhengcheng Zhang, Napersville, OH (US); Qian Liu, Bolingbrook, IL (US)

(73) Assignee: U.S. Government, as Represented by the Secretary of the Army, Adelphi, MD (US)

(21) Appl. No.: 19/542,303

(22) Filed: Feb. 17, 2026

Related U.S. Application Data
(60) Provisional application No. 63/760,950, filed on Feb. 20, 2025.

(51) Int. Cl. H01M 10/26; C07C 209/84; C07C 211/63; C07C 303/40; C07C 303/44; C07C 311/48; H01M 4/42; H01M 4/485; H01M 6/04

(52) U.S. Cl. CPC H01M 10/26; C07C 209/84; C07C 211/63; C07C 303/40; C07C 303/44; C07C 311/48; H01M 4/42; H01M 4/485; H01M 6/045; H01M 2300/0005

(57) ABSTRACT

A molecule, and more particular a salt, formed of an onium cation having at least one ether linkage, and an anion. In particular, it may be tris(3,6-dioxaheptanyl)(methyl)ammonium bis(trifluoromethylsulfonyl)imide, tris(3,6-dioxaheptanyl)(methyl)ammonium bis(fluorosulfonyl)imide (FSI) or tris(3,6-dioxaheptanyl)(methyl)ammonium trifluoromethanesulfonate (OTf). The class of molecules may be used as additive molecules and co-salts in electrolyte solutions, esp. for zinc batteries. Up to about 66 wt. %, the molecule produces significant improvements in the performance of zinc batteries with aqueous electrolytes across multiple metrics. These results are attributed to favorable properties of the ether linkages to suppress dendrite formation and parasitic reactions such as the hydrogen evolution reaction and corrosion.

### RELATED APPLICATION

This application claims benefit to U.S. Provisional Patent Application No. 63/760,950 titled “Onium Cation with Ether Linkages for Zinc Batteries” which was filed on Feb. 20, 2025 (ARL Ref: ARL 23-22P), herein incorporated by reference in its entirety.

### GOVERNMENT INTEREST

The invention described herein may be manufactured, used and licensed by or for the U.S. Government without the payment of royalties thereon.

### RESEARCH AND RELATED DISCLOSURES

Research underlying the invention was funded the Joint Center for Energy Storage Research (JCESR), an Energy Innovation Hub funded by the U.S. Department of Energy (DOE), Office of Science, Basic Energy Sciences (BES), and U.S. Army DEVCOM Army Research Laboratory (ARL). The DOE and ARL were working together under Inter-Agency Agreement No. 89243019SSC000028.

Some aspects of this invention have previously been disclosed by the inventors in the following paper: Glenn R. Pastel et al., “Designing interphases for highly reversible aqueous zinc batteries,” Joule, Vol. 8, 17 Apr. 2024, pp. 1050-1062 (first published Feb. 23, 2024), which was incorporated in and formed a basis of the '950 prov. patent application, and herein incorporated by reference in its entirety for all purposes.

This application is related to U.S. patent application Ser. No. 19/542,252 titled “Pyrrolidinium Cation Having at least one Functional Group for Zinc Batteries” that was filed on Feb. 17, 2026 (ARL Ref: ARL 23-19), herein incorporated by reference in its entirety.

### BACKGROUND OF THE INVENTION

### Field

Embodiments of the present invention are directed to battery chemistry and, more particularly, to electrolyte additives for zinc batteries.

### Description of Related Art

Batteries are ubiquitous on the battlefield in the form of wearables, manned and unmanned vehicles, stationary storage, and many other applications. Zinc batteries provide a safer and more sustainable alternative to conventional lithium-ion batteries. Zinc metal batteries can also achieve high volumetric energy densities which are well suited to military applications with challenging size constraints. Efforts to improve zinc metal anode reversibility in aqueous electrolytes have primarily focused on tailoring Zn²⁺ solvation.

Currently, zinc batteries use a corrosive KOH-based electrolyte. These suffer parasitic reactions including hydrogen evolution, corrosion, and other reactions; they also have limited reversibility and cycle life. As a result, state-of-the-art aqueous zinc electrolytes are not capable of high areal capacities and current densities with reasonable efficiency (1 mAh cm⁻², 1 mA cm⁻², and >90%, respectively). For rechargeable zinc batteries to achieve greater market penetration, electrolytes with significantly better performance must be developed.

In light of the foregoing, improvements for zinc metal battery electrolytes are desired.

### BRIEF SUMMARY OF THE INVENTION

The present invention relates to the application and use of a class of electrolyte additives formed of an onium cation having ether linkages, and an anion for zinc batteries.

Up to about 66 wt. %, the molecule produces significant improvements in the performance of zinc batteries with aqueous electrolytes across multiple metrics. These results are attributed to favorable properties of the ether linkages to suppress dendrite formation and parasitic reactions such as the hydrogen evolution reaction and corrosion.

The onium cation may be selected from the group consisting of: ammonium, sulfonium and phosphonium, as non-limiting examples. The cation may include up to four ether linkages. The ether linkages may include at least one functional group selected from the group consisting of alkyl, alkene, alkenyl, alkynyl, F, CN, carbonate, ester, cyano-substituted alkyl groups, perfluoro, partially-fluorinated groups, ether(s), alcohol, SOₓ, O₂SOR (where R is a Si containing species), O₂SR (where R is fluorine, perfluoro, or other partially fluorinated species bonded to the sulfur atom) and combinations thereof, as non-limiting examples. In one particular embodiment, the at least one functional group is —CH₂—CF₂—CF₃.

The anion may be selected from the group consisting of: bis(trifluoromethane sulfonyl)imide (TFSI), bis(fluorosulfonyl)imide (FSI), trifluoromethanesulfonate (OTf), hexafluorophosphate (PF₆), perchlorate (ClO₄), tetrafluoroborate (BF₄), bis(oxalato)borate (BOB), difluoro(oxalate)borate (DFOB), sulfate (SO₄), nitrate (NO₃), chloride, bromide, fluoride, iodide, acetate, and combinations thereof, as non-limiting examples.

We present a class of novel molecules, and more particularly a class of salt, formed of an onium cation having at least one ether linkage, and an anion. In particular, the molecule may be tris(3,6-dioxaheptanyl)(methyl)ammonium bis(trifluoromethylsulfonyl)imide, tris(3,6-dioxaheptanyl)(methyl)ammonium bis(fluorosulfonyl)imide (FSI) or tris(3,6-dioxaheptanyl)(methyl)ammonium trifluoromethanesulfonate (OTf). The class of molecules may used be as additive molecules and co-salts in electrolyte solutions, esp. for zinc batteries. Up to about 66 wt. %, the molecule produces significant improvements in the performance of zinc batteries with aqueous electrolytes across multiple metrics. These results are attributed to favorable properties of the ether linkages to suppress dendrite formation and parasitic reactions such as the hydrogen evolution reaction and corrosion.

These and other embodiments of the invention are described in more detail, below.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments, including less effective but also less expensive embodiments which for some applications may be preferred when funds are limited. These embodiments are intended to be included within the following description and protected by the accompanying claims.

FIG. 1 shows a schematic illustration of a zinc battery according to embodiments.

FIG. 2(A) shows a 3D rendering of the molecular structure of the tris(3,6-dioxaheptanyl)(methyl)ammonium bis(trifluoromethylsulfonyl)imide. FIGS. 2(B) and 2(C) depict 3D molecular renderings of the chemical structures of the tris(3,6-dioxaheptanyl)(methyl)ammonium cation and the bis(trifluoromethylsulfonyl)imide anion, respectively.

FIG. 3 shows key aspects of the synthesis process for forming TDMA(TFSI) via methylation.

FIG. 4 shows the nuclear magnetic resonance spectra of the product after synthesis of tris(3,6-dioxaheptanyl)(methyl)ammonium bis(trifluoromethylsulfonyl)imide.

FIG. 5 shows galvanostatic cycling tests of Cu∥Zn cells with the TDMA(TFSI).

FIG. 6(A) shows X-ray photoelectron spectra of the zinc metal anode interphase after cycling with the co-salt. FIG. 6(B) shows the FTIR and Raman spectra.

FIG. 7 shows long term cycling stability of Zn∥Zn symmetric cells with and without the co-salt at 1 mA cm⁻² current density and 1 mAh cm⁻² capacity per step.

FIGS. 8(A) and 8(B) are micrographs showing the morphological changes to the baseline electrolyte due to the co-salt from focused-ion beam cross sections viewed by scanning electron microscopy.

### DETAILED DESCRIPTION

We describe the application of a relatively new class of electrolyte additives for use in zinc batteries. Zinc batteries are an alternative to lithium-based batteries. General parameters of zinc batteries are disclosed in Lin Ma et al., “Critical Factors Dictating Reversibility of the Zinc Metal Anode,” Energy Environ. Mater. 2020, 3, 516-521, and corresponding U.S. Patent Application Publication No. 2023/0223595 titled “Heterocyclic Electrolyte Additives for Rechargeable Metal Batteries,” which published Jul. 13, 2023, both of which are herein incorporated by reference in their entireties.

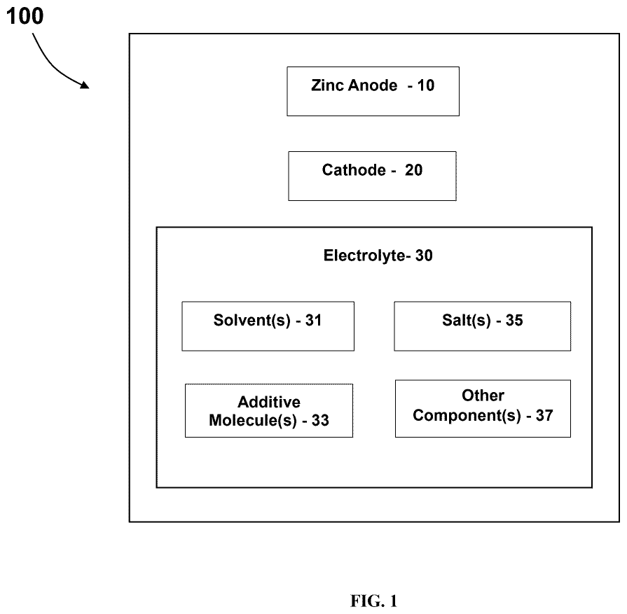

FIG. 1 shows a schematic illustration of a zinc battery 100 according to embodiments. The zinc batteries utilize a zinc metal/alloys anode 10 which offers high volume capacity (e.g., 5884 mAh cm⁻³). The cathode 20 may be formed of a vanadium oxide inorganic compound (HNaV₆O₁₆), a gaseous mixture with oxygen, or an electrochemically-active organic compound such as polyaniline, as non-limiting examples. An electrolyte composition 30 is provided.

The composition 30 includes base electrolyte solvent(s) 31, additive molecule(s) 33, salt(s) 35, and other component(s) 37. The base electrolyte solvent(s) 31 may include one or more liquids commonly used as solvents for electrolytic solutions comprising upwards to nearly 100 weight % ratio. For aqueous solutions, water is used as the sole or primary liquid. Non-aqueous solutions can include alcohols, acids, ethers, carbonates (linear and cyclic), sulfonates, ketones, or hydrocarbons, as non-limiting examples, and can comprise upwards to 100 weight % ratio.

The additive molecule(s) 33 includes at least one molecule formed of an onium cation having at least one ether linkage and an anion. In chemistry, an onium cation is a cation obtained by the protonation of mononuclear parent hydride of a pnictogen (group 15 of the periodic table), chalcogen (group 16), or halogen (group 17). A representative onium cation, and the namesake for the class, is ammonium, NH₄⁺, the protonated derivative of ammonia, NH₃. The onium cation of the additive molecule(s) 33 may be selected from the group consisting of: ammonium, sulfonium and phosphonium, as non-limiting examples. The cation may be a primary, secondary, tertiary, or quaternary onium. Ammonium is a class of ionic compounds with the formula (R)₄N⁺ where R is hydrogen, a linear or cyclic alkyl or alkene, or aryl. Phosphonium is a class of cationic compounds with the formula (R)₄P⁺. Ammonium and phosphonium are both considered Group 15 (pnictogen) onium cations. Sulfonium is a class of cationic compounds with the formula (R)₃S⁺. Sulfonium is considered a Group 16 (chalcogen) onium cation.

The onium cation further includes at least one organic group as a linkage with one or more ether subgroups on the linkage. For example, for an ammonium cation, the ether linkage(s) would attach to the positively-charged N atom. Experiments indicate the favorable properties of the ether linkages suppress dendrite formation and parasitic reactions such as the hydrogen evolution reaction. Ether linkages are bent organic linker groups of the general formula

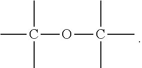

Molecules with four organic linkages with eight total ether groups, or two ethers per linkage, are quite sufficient for purpose of this invention. Primary, secondary, or tertiary oniums may work with more or less ether linkages but would likely require higher loadings or concentrations. Two ether groups per organic linkage is well suited to solvating cations, as represented by the structure of the common solvent dimethoxyethane, with SMILES string COCCOC.

For some molecules, the cation may further comprise at least one functional group, deemed a linkage, attached to and/or including one or more ether groups(s). The at least one functional group may be selected from the group consisting of alkyl, alkene, alkenyl, alkynyl, F, CN, carbonate, ester, cyano-substituted alkyl groups, perfluoro (CFₓ, [CFₓ]_{y}CF_{z}), partially-fluorinated groups, ether(s), alcohol, SOₓ, O₂SOR (where R is a Si containing species), O₂SR (where R is fluorine, perfluoro, or other partially fluorinated species bonded to the sulfur atom), as non-limiting examples. In some embodiments, the ether linkage(s) may be partially or fully fluorinated. In one embodiment, the functional group having an ether linkage is —CH₂—CH₂—O—CH₂—CH₂—O—CH₃. This particular functional group may be advantageous on the basis of two theories. In the first, since the functional group is electrochemically stable but susceptible to decomposition in the presence of a nucleophile/base, such as TFSI⁻ or OH⁻, the reverse Menshutkin reaction and the Hofmann elimination reaction can occur. In the former, a quaternary ammonium cation reacts with a nucleophilic TFSI⁻ or OH⁻ to form R—X and a tertiary amine. The source of OH⁻ in these electrolytes is suspected to be HER, which is still prevalent. In the latter, a β-hydrogen is eliminated from the quaternary ammonium to form an alkene. In a subsequent reaction, especially in relatively acidic electrolytes or in the presence of Zn²⁺ acting as a Lewis acid, the alkene may be hydrated to form an alcohol. The tertiary amine can participate in solid electrolyte interphase (SEI) formation through conversion of dissolved CO₂ to carbonates or hydroxylating exposed ZnO. In the second theory, the functional group is stable and the molecule acts as a steric hindering species against HER during zinc reduction at negatively charged interphases.

The anion of the onium compound may be selected from the group consisting of: bis(trifluoromethane sulfonyl)imide (TFSI), bis(fluorosulfonyl)imide (FSI), trifluoromethanesulfonate (OTf), hexafluorophosphate (PF₆), perchlorate (ClO₄), tetrafluoroborate (BF₄), bis(oxalato)borate (BOB), difluoro(oxalato)borate (DFOB), sulfate (SO₄), nitrate (NO₃), chloride, bromide, fluoride, iodide, acetate, and combinations thereof, for example.

The additive molecule(s) 33 may be tris(3,6-dioxaheptanyl)(methyl)ammonium bis(trifluoromethylsulfonyl)imide (TDMA (TFSI)), 33′ in embodiments. We believe this molecule to be novel and was first reported by us in our 2024 Joule paper, referenced above.

The additive molecule 33′, TDMA (TFSI), has two ether groups on each of three linkages, resulting in good cation solvating properties without restricting solubility or viscosity. This compound is also a room temperature (20° C.) ionic liquid without solvent or solution. Thus, it may be referred to as an “ionic liquid” or also a “co-salt”. The latter term, “co-salt,” can refer to its use with another zinc salt, such as Zn(TFSI)₂. The other zinc salt is responsible for zinc ion conduction while TDMA(TFSI) has a secondary or supporting role, impacting the interphase kinetics and chemistry.

In some embodiments, the additive includes a longer or shorter linear or cyclic functional group with more or less fluorine species. In some embodiments, the other functionals group are similarly varied. In some embodiments, the onium cation with ether groups on some or all organic groups is partially or fully fluorinated, although, with fluorination, the molecule may be less soluble; and/or allow for lower concentrations. The additive molecule(s) 33 may be up to about 66 wt % of the electrolyte composition 30 to substantially improve the performance of zinc metal plating and stripping to resist dendrite formation and raise Coulombic efficiency above 90% Coulombic efficiency.

Electrolyte mixtures that were ionic liquids with a 2:1 ratio of TDMA(TFSI):Zn(TFSI)₂, which is approximately 66 wt %, were tested. However, the viscosity of the mixtures is too high for room temperature applications and they did not cycle at 30° C. Higher concentrations with water, not an ionic liquid, were also tested including 1 and 4 molal concentrations of TDMA(TFSI) with Zn(TFSI)₂. The 4 molal TDMA(TFSI) performed the best of these. The data is presented in Table I, below.

In some embodiments, the additive molecule(s) 33 includes (i) at least one molecule formed of at least an onium cation having at least one ether linkage and an anion, along with (ii) an additional additive molecule including a pyrrolidinium cation, with at least one functional (i.e., reactive) group, and an anion as disclosed in the related '252 patent application (ARL Ref: ARL 23-19). There may be tradeoffs in terms of performance and optimization may be further required. For instance, adding TDMA(TFSI) to PfmPyr(TFSI) resulted in longer Zn|Zn cycle life (less dendrite growth), but PfmPyr(TFSI) contributes a lower overpotential. Other potential combinations of the different additive molecule classes may indeed be advantageous and/or synergistic for zinc batteries. The concentrations thereof when combined may need to be varied compared to their use individually.

Salt(s) 35 may include various dissociated salts, such as imides, sulfates, and hydroxides, as non-limiting examples. The composition of the co-salt, synthesis process, baseline electrolyte, or electrodes can be varied within the bounds of this invention. In some embodiments, the co-salt includes longer or short ether linkages, i.e. Simplified Molecular Input Line Entry System (SMILES) strings: COCOCC, CCOCCOCC, COCCOCCOCC, with more or less halide species, i.e. F or Cl. The salt(s) may also include at least one metal salt. The metal salt may have a cation selected from the group consisting of: zinc (Zn), lithium (Li), sodium (Na), potassium (K), iron (Fe), magnesium (Mg), calcium (Ca), vanadium (V), aluminum (Al), and any combination thereof. And the metal salt may have an anion selected from the group consisting of: bis(trifluoromethane sulfonyl)imide (TFSI), bis(fluorosulfonyl)imide (FSI), trifluoromethanesulfonate (OTf), hexafluorophosphate (PF₆), perchlorate (ClO₄), tetrafluoroborate (BF₄), bis(oxalato)borate (BOB), difluoro(oxalato)borate (DFOB), sulfate (SO₄), nitrate (NO₃), chloride, bromide, fluoride, iodide, acetate, and any combination thereof. The concentration of the metal salts may be between 0.1 molal and 30 molal in the electrolyte composition.

Other component(s) 37 may optionally be included in the electrolyte solution at low concentrations. (e.g., ≤5% by weight). This is determined partially by solubility since the hydrophobic groups on the cation limit dissociation in aqueous media and are therefore highly dependent on the solvent(s). Polymer(s), such as polyethylene glycol, polyethylene oxide, poly(vinylidene fluoride-co-hexafluoropropylene), may be incorporated into the electrolyte to provide benefits. One or more of hydroxides, chlorides, sulfates, zinc acetate, zinc nitrate, zinc trifluoromethanesulfonate, and zinc bis(trifluoromethylsulfonyl)imide may be further added to the baseline electrolyte to further provide conductivity, SEI reagents, and a zinc source. Anion chemistry impacts many properties as well.

Moreover, the electrolyte can include catholyte or anolyte or can be used in a flow battery configuration. In some embodiments, the additive is used for a zinc-air battery, zinc-ion battery, zinc hybrid supercapacitor, zinc-halide battery, zinc-iron redox flow battery, zinc-cerium redox flow battery, zinc-silver battery, zinc-organic cathode battery, zinc-MnO₂, or a zinc hybrid battery with two active cations.

The exact composition of the additive, synthesis process, baseline electrolyte, and/or electrodes in the zinc battery 10 can be varied within the bounds of this invention.

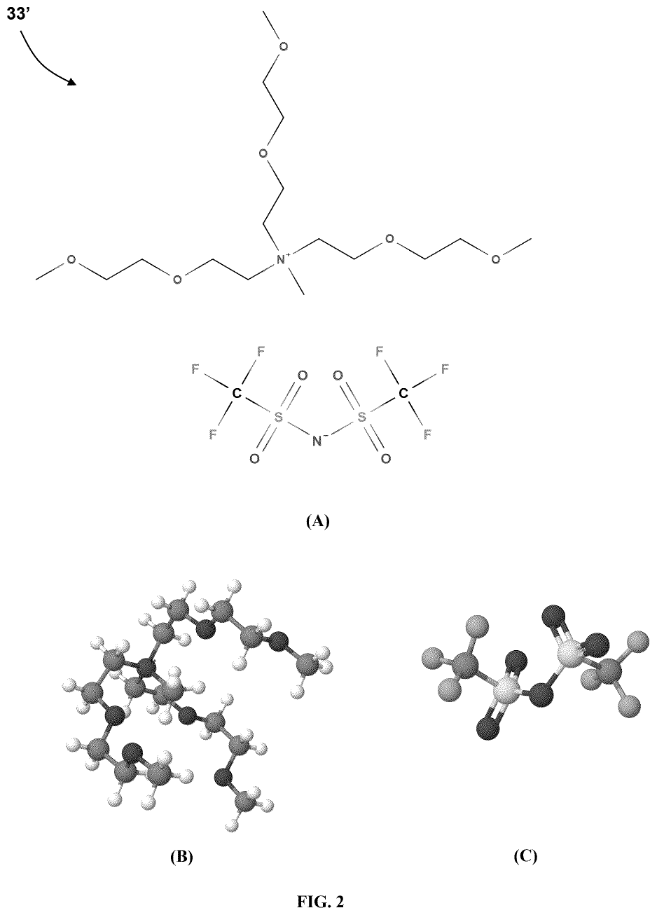

FIG. 2(A) depicts the molecular formula of the tris(3,6-dioxaheptanyl)(methyl)ammonium bis(trifluoromethylsulfonyl)imide. FIGS. 2(B) and 2(C) depict 3D molecular renderings of the chemical structures of the tris(3,6-dioxaheptanyl)(methyl)ammonium cation and the bis(trifluoromethylsulfonyl)imide anion, respectively. The chemical formula is (C₁₆H₃₆NO₆)⁺(C₂F₆NO₄S₂)⁻. The Simplified Molecular Input Line Entry System (SMILES) string for this molecule is as follows: cation=COCCOCC[N+](C)(COCCOCC)COCCOCC and anion=C(F)(F)(F)S(═O)(═O)[N—]S(═O)(═O)C(F)(F)F}. The molar mass of the compound is 618.60 amu with the cation weighing 338.46 amu and the TFSI anion weighing 280.14 amu.

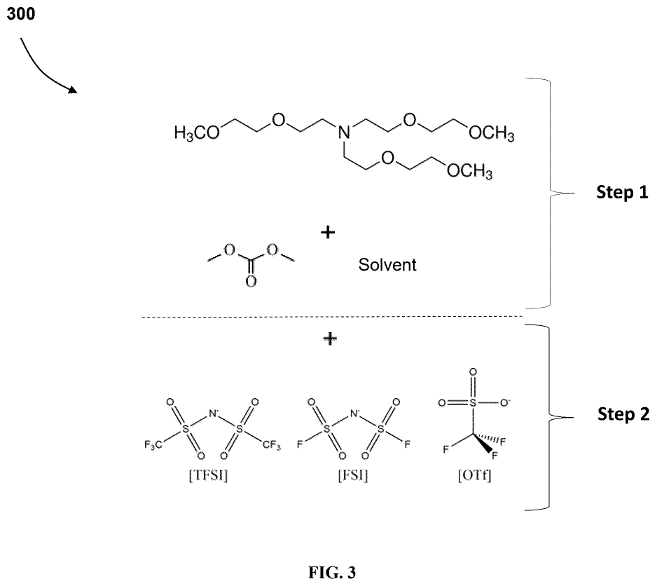

FIG. 3 shows key aspects of the synthesis process 300 for forming TDMA(TFSI) via methylation. The tris[2-(2-methoxyethoxy)ethyl]ammonium bis(trifluoromethylsulfonyl)imide TDMA(TFSI) was prepared via a two-step reaction (schematic). In Step 1, tris[2-(2-methoxyethoxy)ethyl]amine, dimethyl carbonate, and methanol were added into a pressure reactor. Then, the pressure reactor was heated up to 120° C. for 12 h with constant stirring inside not exceeding 100 psi. In Step 2, bis(trifluoromethanesulfonyl)imide (TFSI) aqueous solution was slowly added dropwise to the product of the first step under constant stirring until the pH is neutral. Then, reflux was carried out by mixing the product with 10 g active carbon in an acetonitrile solution environment for 2 h to remove residue impurities. Active carbon was removed by filtering the mixture afterward. The final pure product was obtained by drying the filtered mixture at 60° C. under vacuum for 24 h.

Different amide anions (e.g., TFSI, FSI, OTf) can easily be substituted in the synthesis by adding a different acid precursor in Step 2. The corresponding acids are (aqueous solutions with dilute concentrations of) bistriflimidic acid (for TFSI), bis(fluorosulfonyl)imidic acid (for FSI), and triflic acid (for OTf). Chemists will understand other classes of anions can be added with other suitable reactants and/or precursors.

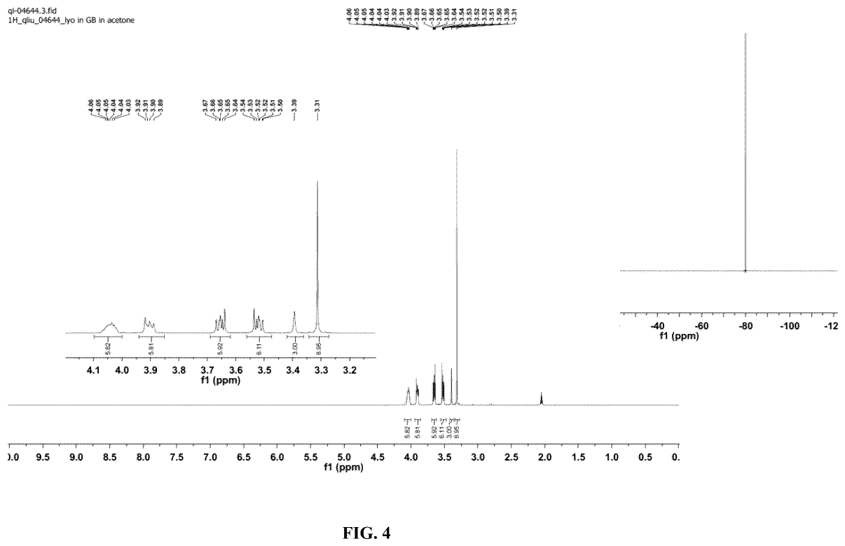

FIG. 4 shows the nuclear magnetic resonance spectra of the product after synthesis of tris(3,6-dioxaheptanyl)(methyl)ammonium bis(trifluoromethylsulfonyl)imide. The NMR spectra data confirms the purity of the compound and is critical for researchers interested in replicating the synthesis.

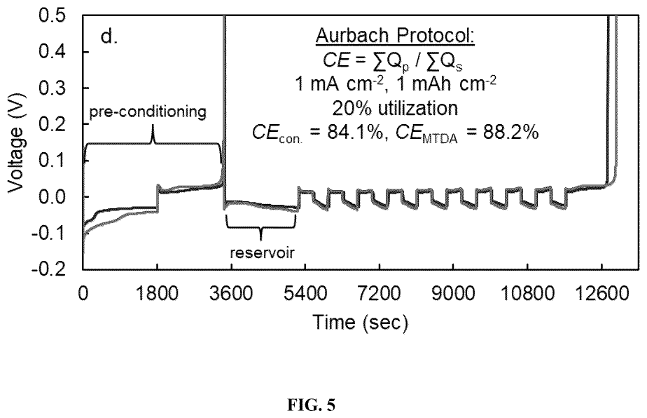

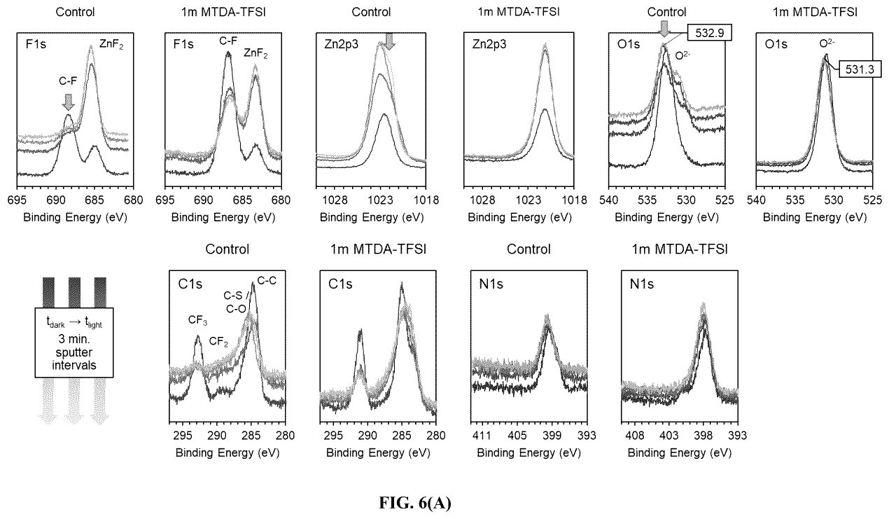

FIG. 5 shows galvanostatic cycling tests of Cu∥Zn cells with the co-salt, derived from the Aurbach protocol (doi: 10.1149/1.2096425), with a 5 mAh cm⁻² reservoir and 20% utilization. The cycling tests indicate an increase in Coulombic efficiency from 84.1% up to 99% relative to a baseline electrolyte without the ammonium co-salt (Table I and FIG. 5, additive in blue, baseline in black). Our experiments indicate that a change to the zinc metal interphase occurs due to the co-salt with less zinc oxide and more fluorinated species (FIG. 6(A)). However, exploration into co-salts with other anions or functional groups (i.e. alkyl, alkene, alkenyl, alkynyl, F, CN, carbonate, ester, cyano-substituted alkyl groups, perfluoro (CFₓ, [CFₓ]_{y}CF_{z}), partially fluorinated, ether(s), alcohol, SOₓ, O₂SOR (R is a Si containing species), SO₂R (R is fluorine, perfluoro, or partially fluorinated off of the sulfur atom)) could produce better performance. Raman and infrared spectroscopy help reveal the solvation behavior and reaction mechanisms of the co-salt (FIG. 6(B)).

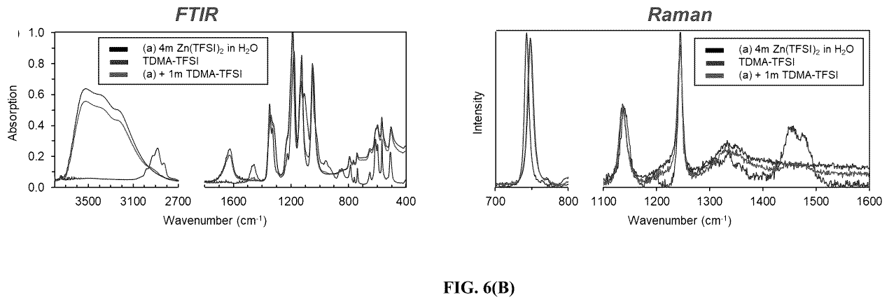

Long term cycling tests of symmetric Zn∥Zn cells show outstanding stability and resilience against dendrites, corrosion, and irreversibility (FIG. 7). A small amount of the ammonium co-salt results in over a ten-fold improvement over the 4 m Zn(TFSI)₂—H₂O baseline electrolyte. This also indicates a substantial improvement over the standard KOH based electrolyte, which is unstable within 50 hours of cycling under the same conditions.

Table I, below, shows results from Coulombic efficiency tests of
Cu ∥ Zn cells with high loadings of the TDMA(TFSI) co-salt
with only a single glass fiber separator layer and constructed
in an open atmosphere environment. Coulombic efficiency improves
with the concentration of TDMA(TFSI) while viscosity increases.

Loading

| Electrolyte | 0.5 mA/cm² | 1 mA/cm² | 5 mA/cm2 |
|---|---|---|---|
| 4.0 m Zn(TFSI)₂ H₂O | 79% | 84% | Short |
| +2 wt % TDMA(TFSI) | 87% | 89% | Short |
| +1.0 m TDMA(TFSI) | 87% | 88% | 97% |
| +4.0 m TDMA(TFSI) | 91% | 99% | |

Notably, all the TDMA(TFSI) embodiments of the present invention show improved performance under high loadings compared to the control.

FIG. 6(A) shows X-ray photoelectron spectra of the zinc metal anode interphase after cycling with the co-salt which indicates significant changes in the F, Zn, and O spectra with depth profiling. The co-salt increases C—F and reduces ZnO/Zn(OH)₂ shoulder peaks. FIG. 6(B) shows the FTIR and Raman spectra. The broad O—H stretching band indicates the degree of hydrogen bonding and can be deconvoluted to three populations: “network water” (~3205 cm⁻¹), “intermediate water” (~3433 cm⁻¹), and multimer water” (~3560 cm⁻¹). Generally, more extensive hydrogen bonding leads to longer O—H stretching and a shift to lower wavenumbers. A quantitative deconvolution on these three components shows that the addition of TDMA(TFSI) hardly impacts the hydrogen bonding network of the baseline electrolyte. The Raman spectra further confirms the SO₃ and CF₃ vibrational bands of the co-salt.

FIG. 7 shows long term cycling stability of Zn∥Zn symmetric cells with and without the co-salt at 1 mA cm⁻² current density and 1 mAh cm⁻² capacity per step. Notably, additive loading of the TDMA(TFSI) (2 wt %) increases the overpotentials observed in the voltage profile, but also results in over a ten-fold improvement in cycle lifetime.

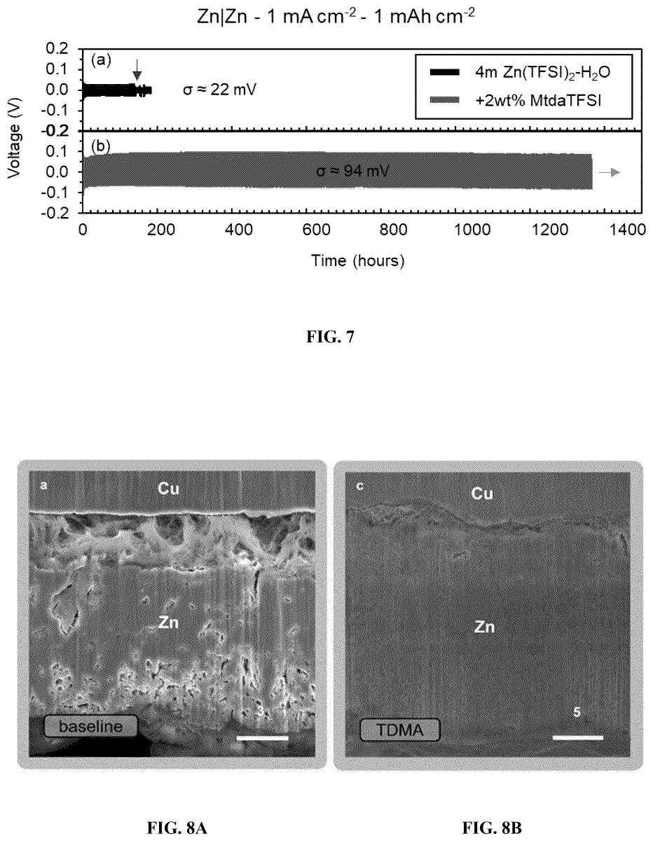

FIGS. 8(A) and 8(B) are micrographs showing the morphological changes to the baseline electrolyte due to the co-salt from focused-ion beam cross sections viewed by scanning electron microscopy. The deposit with the TDMA(TFSI) co-salt is much denser than the deposit from the control electrolyte and maintains better attachment to the copper current collector. The improved homogeneity also aligns with the reduced dendrite susceptibility, relative to the control.

The present invention enables major improvements in cycle life and efficiency compared to state-of-the-art aqueous zinc electrolytes. Coulombic efficiencies above 99% are feasible with ten-fold improvement in cycle life. These metrics represent a huge step toward commercially viable zinc metal batteries. Additionally, the synthesis of this class of co-salts is straightforward as evidenced from the present invention. Flexibility in mass loading (e.g., up to 66 wt %) is also advantageous to realize improvements in performance. Furthermore, this class of additives represents a promising template for further optimization of co-salt chemistries.

This technology is appealing for commercial applications because it does not require modifications to the manufacturing process and it addresses a key challenge (dendrites) associated with zinc metal batteries. Importantly, this co-salt can be used in combination with other minority components in the electrolyte for distinct and correlative effects, resulting in substantial improvements to performance.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present disclosure and its practical applications, and to describe the actual partial implementation in the laboratory of the system which was assembled using a combination of existing equipment and equipment that could be readily obtained by the inventors, to thereby enable others skilled in the art to best utilize the invention and various embodiments with various modifications as may be suited to the particular use contemplated.

While the foregoing is directed to embodiments of the present invention, other and further embodiments of the invention may be devised without departing from the basic scope thereof, and the scope thereof is determined by the claims that follow.

1. A zinc battery cell comprising:
an anode formed of zinc metal or zinc alloy;
a cathode; and
an electrolyte composition comprising an additive molecule formed of an onium cation having at least one ether linkage, and an anion.

2. The zinc battery cell of claim 1, wherein the cathode is selected from the group consisting of: a vanadium oxide inorganic compound (HNaV₆O₁₆), a gaseous mixture with oxygen, and an electrochemically-active organic compound.

3. The zinc battery cell of claim 1, wherein the electrolyte composition further comprises one or more solvents, one or more salts, one or more additional additives, or any combination thereof.

4. The zinc battery cell of claim 1, wherein the additive molecule comprises up to about 66 wt. % of the electrolyte composition.

5. The zinc battery cell of claim 1, wherein the Coulombic efficiency of the electrolyte composition in the battery cell is at least 90%.

6. The zinc battery cell of claim 1, wherein the concentration of the one or more salts is between 0.1 molal and 30 molal in the electrolyte composition.

7. The zinc battery cell of claim 1, wherein water comprises a main solvent in the electrolyte composition.

8. The zinc battery of claim 1, wherein the onium cation is selected from the group consisting of: ammonium, sulfonium and phosphonium.

9. The zinc battery cell of claim 1, wherein the cation further comprises at least one functional group is selected from the group consisting of alkyl, alkene, alkenyl, alkynyl, F, CN, carbonate, ester, cyano-substituted alkyl groups, perfluoro, partially-fluorinated, ether(s), alcohol, SOₓ, O₂SOR (where R is a Si containing species), and O₂SR (where R is fluorine, perfluoro, or other partially fluorinated bonded to the sulfur atom).

10. The zinc battery cell of claim 9, wherein at least one functional group is —CH₂—CH₂—O—CH₂—CH₂—O—CH₃.

11. The zinc batter of claim 1, wherein the cation has one to three ether linkages.

12. The zinc batter of claim 1, wherein the cation has four ether linkages.

13. The zinc battery of claim 1, wherein the anion is selected from the group consisting of: bis(trifluoromethane sulfonyl)imide (TFSI), bis(fluorosulfonyl)imide (FSI), trifluoromethanesulfonate (OTf), hexafluorophosphate (PF₆), perchlorate (ClO₄), tetrafluoroborate (BF₄), bis(oxalato)borate (BOB), difluoro(oxalato)borate (DFOB), sulfate (SO₄), nitrate (NO₃), chloride, bromide, fluoride, iodide, acetate, and combinations thereof.

14. The zinc battery of claim 1, wherein the additive molecule is tris(3,6-dioxaheptanyl)(methyl)ammonium bis(trifluoromethylsulfonyl)imide, tris(3,6-dioxaheptanyl)(methyl)ammonium bis(fluorosulfonyl)imide (FSI) or tris(3,6-dioxaheptanyl)(methyl)ammonium trifluoromethanesulfonate (OTf).

15. The zinc battery of claim 1, configured as a rechargeable battery or a primary battery.

16. A method of forming a zinc battery cell comprising
providing or forming an anode comprising zinc metal or zinc alloy;
providing or forming a cathode; and
providing or forming an electrolyte composition comprising an additive molecule formed of an onium cation having at least one ether linkage and an anion.

17. A molecule, or salt, of tris(3,6-dioxaheptanyl)(methyl)ammonium bis(trifluoromethylsulfonyl)imide, tris(3,6-dioxaheptanyl)(methyl)ammonium bis(fluorosulfonyl)imide (FSI) or tris(3,6-dioxaheptanyl)(methyl)ammonium trifluoromethanesulfonate (OTf).

18. An electrolyte composition comprising the molecule or salt of claim 17.

19. A method of synthesizing the tris(3,6-dioxaheptanyl)(methyl)ammonium bis(trifluoromethylsulfonyl)imide or salt thereof according to claim 17, the method comprising:
combining tris[2-(2-methoxyethoxy)ethyl]amine, dimethyl carbonate, and a solvent were added into a pressure reactor at a pressure no greater than 100 psi;
heating the pressure reactor to about 120° C. for 12 h with constant stirring inside;
adding bis(trifluoromethanesulfonyl)imide H(TFSI) aqueous solution to the heated solution under constant stirring until the pH becomes neutral;
carrying out reflux by mixing the product with about 10 g active carbon in an acetonitrile solution environment for 2 h to remove residue impurities;
remove the active carbon by filtering the mixture afterward; and
drying the filtered mixture at 60° C. under vacuum for 24 h.
